(19) United States
(12) Patent Application Publication
(10) Pub. No.: US 2008/0228045 A1
(43) Pub. Date: Sep. 18, 2008

(54) Multiprotocol Wireless Medical Monitors and Systems

(76) Inventors: Tia Gao, Ellicott City, MD (US); Leo Selavo, Riga (LV)

Correspondence Address: PATENTBEST, 4600 ADELINE ST., #101, EMERYVILLE, CA, 94608, US

(21) Appl. No.: 12/035,664

(22) Filed: Feb. 22, 2008

Related U.S. Application Data
(60) Provisional application No. 60/891,437, filed on Feb. 23, 2007.

(51) Int. Cl. A61B 5/00

(52) U.S. Cl. 600/301

(57) ABSTRACT

A wireless medical monitoring system and medical monitoring devices adapted to communicate using a plurality of wireless protocols and networks. For each transmission of data, a wireless protocol or network is selected based on the properties of the available protocols and networks and the nature of the data that is to be transmitted. Thus, the medical system and devices can move seamlessly from one context and location to another. The medical devices may also include additional features, such as detection of improperly positioned or disconnected sensors and auditory and/or visual prompting of the patient to correct the problem. In some embodiments, the medical monitoring devices may comprise body area networks of individual sensors communicating and cooperating with one another wirelessly.

### CROSS-REFERENCE TO RELATED APPLICATIONS

This application claims priority to, and the benefit of, U.S. Provisional Patent Application No. 60/891,437, filed on Feb. 23, 2007, the contents of which are incorporated by reference herein in their entirety.

### BACKGROUND OF THE INVENTION

1. Field of the Invention

The invention relates generally to the field of medical sensors, and more particularly to medical sensors equipped for wireless transmission.

2. Description of Related Art

Electronic vital sign monitoring has long been an important part of the standard of care for most hospitalized patients and for some patients in non-hospital settings. Electrocardiogram (ECG), electroencephalogram (EEG), heart rate, blood pressure, pulse oximetry, body temperature, blood chemistry and other vital signs and indicators gathered by electronic monitoring are used as diagnostic tools, they are used to determine whether a patient's condition is improving or worsening, and they are used for triage, to allocate medical care and personnel to the neediest patients.

Traditionally, if a patient was to be monitored, the patient would be connected by wires or other electrical leads to sensors and instrumentation located at the bedside, and monitoring could take place only so long as the patient was in bed and immediately proximate to the monitoring instrumentation. However, as technologies have improved and become more portable, monitoring has become easier, and patients can now be monitored in a variety of settings. For example, emergency medical technicians (EMTs) now carry portable 12-lead ECG machines, and pulse oximetry equipment has become so simple and portable that even the lowest-level first responders are being taught to use it.

In the last few years, wireless communication technology has pervaded almost every aspect of life. Cellular telephones are ubiquitous, automobiles come equipped with Global Positioning System (GPS) receivers, and laptops feature wireless networking adapters. This revolution in wireless communications has also slowly affected the medical field—for example, some monitors can now transmit vital signs wirelessly.

However, there are multiple wireless standards, each with its own strengths, weaknesses, and technical requirements, and each incompatible with the others. If, for example, EMTs connected a patient to a wireless monitoring system within an ambulance during transport to the hospital, the patient may need to be disconnected from that system and connected to a different system once he or she reaches the hospital. Such problems are counterproductive and can make wireless monitoring less useful.

### SUMMARY OF THE INVENTION

One aspect of the invention relates to medical monitors comprising one or more sensors and one or more wireless interface units. The medical monitors may select an appropriate wireless interface and/or protocol for each transmission of data based on environmental conditions, sensor conditions, or the nature of the medical data to be transmitted. In some embodiments, the medical monitors may comprise networks of individual sensors, each sensor having one or more associated wireless interface units, the sensors communicating and cooperating with each other wirelessly.

Other aspects of the invention relate to medical monitoring systems capable of using multiple wireless protocols to communicate, depending on environmental conditions, sensor conditions, and the nature of the medical data to be communicated.

Other aspects, features, and advantages of the invention will become clear in the description that follows.

### BRIEF DESCRIPTION OF THE DRAWINGS

The invention will be described with respect to the following drawing figures, in which like numerals represent like features throughout the figures, and in which:

FIG. 1 is an illustration of a medical monitoring system according to one embodiment of the present invention;

FIG. 2 is a schematic illustration of the components of a medical monitoring device according to one embodiment of the invention;

FIG. 3 is a schematic illustration of the components of a medical monitoring device according to another embodiment of the invention;

FIG. 4 is a perspective view of a medical monitoring device according to one embodiment of the invention;

FIG. 5 is a flow diagram illustrating the tasks of selecting and switching between wireless networks in medical monitoring systems according to embodiments of the invention; and

FIG. 6 is an illustration of a medical monitoring system according to another embodiment of the invention, in which individual sensors interoperate to form a wireless body area network.

### DETAILED DESCRIPTION

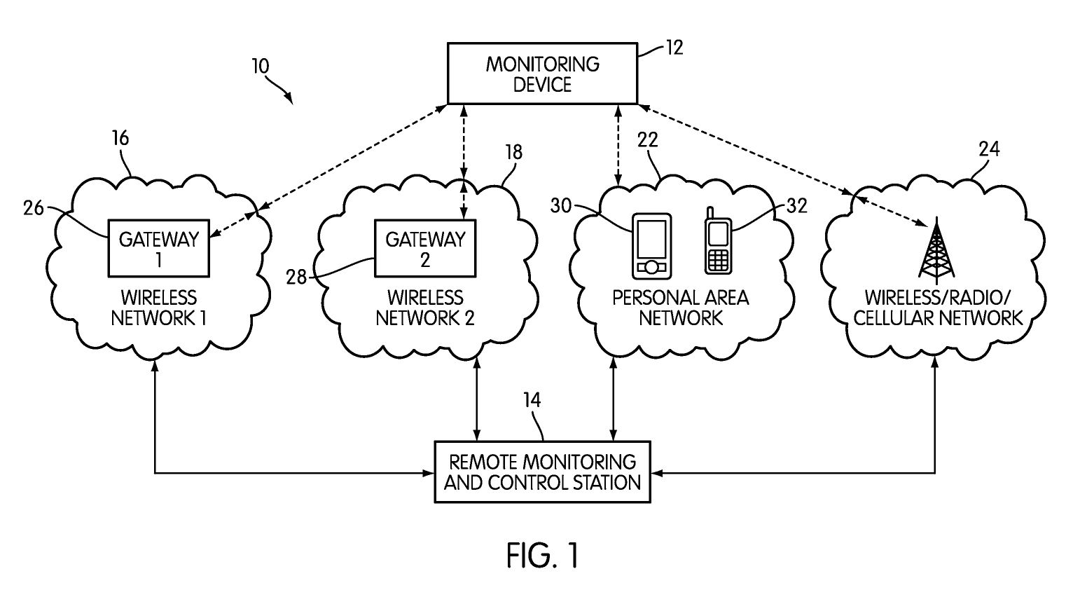

FIG. 1 is an illustration of a medical monitoring system, generally indicated at 10. The medical monitoring system 10 includes one or more medical monitoring devices 12 (one is shown in the illustration of FIG. 1, although any number may be used) and one or more remote monitoring and control stations 14. Each monitoring device 12 includes one or more medical sensors designed to sense some aspect of the condition of a patient. Furthermore, each monitoring device 12 is designed, sized, and adapted to be portable, and has additional features that will be described below in more detail.

Although some aspects of the invention will be described below with respect to medical monitoring in hospital and pre-hospital environments, the medical monitoring system 10 of the present invention and its components may be used in a variety of settings, and generally in any setting in which continuous medical information would be helpful. Other examples of suitable uses and settings include long-term monitoring in rehabilitative (post-hospital) settings and monitoring of homebound patients. The medical monitoring system 10 may also be used to monitor those in occupations that have a high degree of risk of injury. For example, the medical monitoring system 10 may be used to monitor soldiers on the battlefield. It should also be understood that the term “monitoring” is used only for convenience in description; in some embodiments, the medical monitoring system 10 may be used to deliver medical interventions and care, and thus, its role may not be limited strictly to monitoring.

Each of the monitoring devices 12 in the medical monitoring system 10 is in communication with a remote monitoring and control station 14 that provides users, such as medical personnel, access to the data on the conditions of the individual patients that is generated by the medical monitoring devices 12. The communication between the monitoring devices 12 and the remote monitoring and control station 14 is wireless. Moreover, each monitoring device 12 is equipped to communicate with the remote monitoring and control station 14 using several different wireless protocols and wireless networks and is adapted to choose different wireless protocols and networks for different types of transmissions and different situations, based on the properties of the wireless protocols and networks and the nature of the medical data to be transmitted.

The wireless networks and protocols through which and with which the monitoring devices 12 communicate with the remote monitoring and control station 14 may be any wireless networks and protocols known in the art, and the monitoring devices 12 can be equipped to use any number of different wireless networks and protocols to transmit data.

A number of wireless communication protocols and networks exist. Some of these protocols are intended to establish local and wide area networks between general-purpose computers, like WiFi (IEEE 802.11g-2003, “IEEE Standard for Information technology—Telecommunications and information exchange between systems—Local and metropolitan area networks—Specific requirements—Part 11: Wireless LAN Medium Access Control (MAC) and Physical Layer (PHY) specifications—Amendment 4: Further Higher-Speed Physical Layer Extension in the 2.4 GHz Band,” IEEE, 2003) and WiMax (IEEE 802.16e-2005, “IEEE Standard for Local and metropolitan area networks Part 16: Air Interface for Fixed and Mobile Broadband Wireless Access Systems Amendment for Physical and Medium Access Control Layers for Combined Fixed and Mobile Operation in Licensed Bands,” IEEE, 2005). These two protocols, WiFi and WiMax, are designed for high-bandwidth applications that require large amounts of data to be transmitted in a short time period. However, they require relatively large amounts of power to transmit and receive.

Other wireless protocols are designed for wireless personal area networks, like the Bluetooth protocol (IEEE 802.15.1-2002, “Wireless MAC and PHY Specifications for Wireless Personal Area Networks (WPANs™)” IEEE, 2002.) Yet other wireless protocols are designed for wireless personal area networks in which the components in communication will not require a large bandwidth (i.e., the components will not need to transmit large amounts of data in a small amount of time), like the IEEE 802.15.4-2002 standard. (IEEE 802.15.4-2002, “Wireless Medium Access Control (MAC) and Physical Layer (PHY) Specifications for Low Rate Wireless Personal Area Networks (LR-WPANs)” IEEE, 2002.) All of the standards referenced herein are hereby incorporated by reference in their entireties.

In addition to the various wireless networking standards that are commonly used to connect general-purpose computers, embodiments of the invention may use other radio frequency bands and other types of protocols. For example, the monitoring device 12 may be equipped to communicate via a cellular telephone network. Additionally, in order to avoid interference in the unlicensed, general-purpose frequency bands, the monitoring device 12 may be equipped to use the wireless medical telemetry services (WMTS) band at 608 MHz.

There are also a number of frequency bands set aside for the use of municipal and regional emergency services workers, and radio communication protocols exist for accessing those radio communication networks. Some embodiments of the invention may communicate using those frequency bands and protocols. For example, particularly if the monitoring device 12 is used for triage in the field (e.g., during a multiple casualty incident), communication over those emergency services radio networks may be desirable.

Thus, embodiments of the invention may be made to function with substantially any sort of wireless network, communication protocols, or radio frequency band. By way of example, FIG. 1 illustrates four types of wireless networks 16, 18, 22, 24 with which the monitoring device 12 is equipped to communicate.

Wireless network 16 may be assumed to be an IEEE 802.11a/b/g wireless network (also called a WiFi network) having a gateway 26 (also called a wireless access point or base station) with which the monitoring device 12 communicates to send data over the wireless network 16. Therefore, wireless network 16 may be used to transmit data that requires a relatively high bandwidth. However, transmitting data via wireless network 16 may require more power than transmitting via other kinds of networks.

Wireless network 18 may be assumed to be an IEEE 802.15.4 wireless network, with its own gateway or repeater, indicated at 28. Therefore, wireless network 18 may be used to transmit low-bandwidth data using relatively small amounts of power. In some embodiments, the ZigBee communication standards may be implemented. In that case, the monitoring device 12 would be a ZigBee end device, the gateway 28 would be a ZigBee router, and the processing and display device 14 would be a ZigBee coordinator.

Wireless network 22 may be assumed to be an IEEE 802.15.1, Bluetooth, or Personal Area Network. In addition to communicating with the remote monitoring and control station 14, other devices 30, 32 may be included in the personal area network. For example, data from the monitoring device 12 could be sent directly to a physician's personal digital assistant (PDA) 30 or cellular telephone 32 in addition to being sent to the remote monitoring and control station 14. A personal area network like wireless network 22 may be particularly useful if the remote monitoring and control station 14 is in close proximity, for example, as it might be in an ambulance.

Wireless network 24 is representative of a number of radio communication networks. These include the types of WMTS bands, emergency services radio communication bands and networks described above, as well as cellular telephone networks and paging networks. This type of wireless network 24 may be particularly useful at longer ranges, or in locations where other types of networks do not exist.

In some embodiments, the monitoring devices 12 may use more than one wireless communication protocol concurrently. For example, some of the medical sensors may be in communication with the monitoring device 12 wirelessly through a personal area network. While receiving data from those medical sensors, the monitoring device 12 may choose another protocol to transmit data to the remote monitoring and control station 14. Similarly, a monitoring device 12 may transmit data directly to an attending physician's PDA 30 or other personal computing device using a personal area network such as wireless network 22 or a cellular telephone network such as wireless network 24 while concurrently transmitting data to the remote monitoring and control station 14 through wireless network 16 or 18.

Additionally, the monitoring devices 12 may use more than one wireless network 16, 18, 22, 24 concurrently or independently for purposes of redundancy, in order to ensure that a particular monitoring device 12 is always in contact with the remote monitoring and control station 14 by way of at least one wireless network 16, 18, 22, 24.

In some embodiments, the monitoring devices 12 may be connected to the remote monitoring and control station 14 without a gateway; that is, the wireless network may comprise the monitoring devices 12 and the remote monitoring and control station 14 without intervening equipment. As will also be described below, in some embodiments, the monitoring devices 12 may be connected directly to the remote monitoring and control station 14 using a wired connection.

In general, one advantage of the medical monitoring system 10 and its monitoring devices 12 is that the monitoring devices 12 may move from one location and context to another seamlessly, assuming that there is always some wireless network 16, 18, 22, 24 to connect the monitoring devices 12 to the remote monitoring and control station 14. As used here, the term “seamless” refers to a connection in which at least one of the following conditions is true: (1) the monitoring devices 12 switch from one wireless protocol or network substantially without user or patient intervention, based on the properties of the available wireless protocols and networks and the type of data to be communicated; and (2) in making a switch between one wireless protocol or network and another, essentially no patient data is lost. The nature of the seamless communication will be described in greater detail below with respect to the method of operation of the monitoring devices 12. Of course, the ability of the monitoring devices 12 to operate seamlessly depends in large part on the reliability of the wireless networks and protocols it uses to communicate, and situations in which no reliable wireless network or protocol is available will occasionally arise.

The remote monitoring and control station 14 may be any general purpose or special purpose computing system capable of performing the functions described herein. Moreover, although shown as a single entity in FIG. 1 for ease of illustration and description, the remote monitoring and control station 14 may comprise one or more general purpose or special purpose computing systems operating cooperatively or independently. If the remote monitoring and control station 14 comprises multiple computing systems, those systems may be physically located in the same place or geographically distributed. In one embodiment, for example, the processing and display functions of the remote monitoring and control station 14 may be separated. More specifically, data from the monitoring devices 12 may be received by a central server that stores and indexes the data and shown on one or a plurality of display monitors or terminals in communication with that central server. Display monitors may, for example, be located at the patient's bedside, at nurses' and central monitoring stations, and in physician offices, as well as in other locales.

Furthermore, there may be more than one remote monitoring and control station 14. For example, during transport of the patient to the hospital, the remote monitoring and control station may be a laptop computer, and when the patient arrives at the hospital, computing systems under the aegis of the hospital may take over the functions of the remote monitoring and control station 14. Additionally, data from a portable remote monitoring and control station 14 may be transferred to another remote monitoring and control station 14 through a network or by other means.

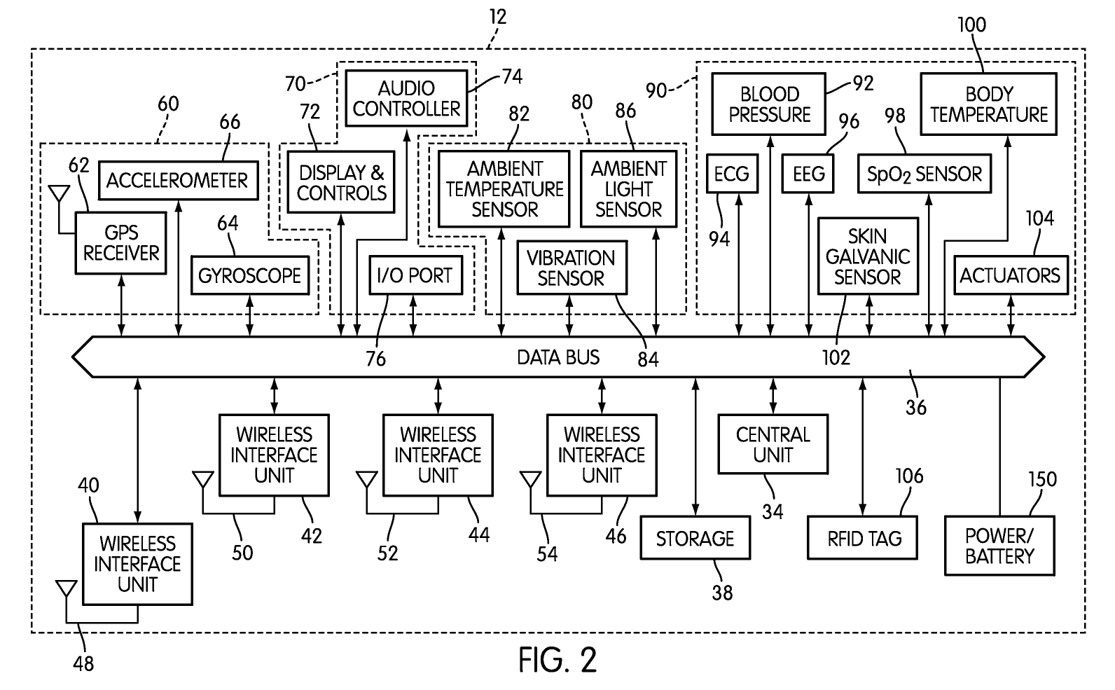

FIG. 2 is a functional block diagram of one embodiment of the monitoring device 12. Within the monitoring device 12, a central unit 34 is responsible for collecting and processing data from the various sensors and other components, deciding which wireless protocol or network to use at any given time and for any given purpose, controlling the flow of data to and from the wireless networks 16, 18, 22, 24, and performing other administrative tasks during the operation of the monitoring device 12. The central unit 34 may be a microprocessor, an application-specific integrated circuit (ASIC), or any other component capable of performing the functions described herein. Moreover, several integrated circuits or components may cooperatively perform the functions of the central unit 34. In one exemplary embodiment, the central unit 34 may comprise a Texas Instruments TI MSP430 microcontroller (Texas Instruments, Dallas, Tex.). However, other types of devices may be used in other embodiments.

Connected to the central unit 34 is a data bus 36 which carries information between the central unit 34 and the other components of the monitoring device 12. It should be understood that some or all of the elements and devices shown in FIG. 2 as connected to the data bus 36 may require signal conditioning and filtering equipment, analog-to-digital converters, and other similar devices in order to connect to the data bus 36. Those devices are omitted from FIG. 2 in order to ensure clarity in illustration, although the monitoring device 12 may include them, connected between the respective devices and the data bus 36, if necessary or desirable. Alternatively, in some cases, the analog-to-digital conversion and signal conditioning components may be integrated into the respective controllers for the elements and devices. If necessary, one or more digital-to-analog converters may also be provided to convert digital signals from the data bus 36 into analog signals for the use of analog devices and elements, if any are provided.

The central unit 34 may include some amount of internal storage memory. For example, the TI MSP430 microprocessor includes 10 kb of random access memory (RAM) and another 48 kb of programmable flash memory. However, as shown in FIG. 2, storage 38 is also connected to the data bus 36 so as to be in communication with the central unit 34 and the other components of the monitoring device 12. As used here, the general term “storage” refers broadly to any type of electronic memory usable in the monitoring device 12, including RAM, read-only memory (ROM), electronically erasable and programmable memory, flash memory, and removable storage media, including flash drives, optical drives, and magnetic media (e.g., hard disk drives and floppy drives). In most embodiments, the storage 38 will comprise a number of different types of memory, including, for example, RAM, flash memory, and, optionally, a hard disk drive. The amount of RAM provided in the monitoring device will depend on a number of factors, including the nature of the wireless networks 16, 18, 22, 24 with which the monitoring device 12 is adapted to communicate, the nature of the sensors included in the monitoring device 12 and their memory requirements, and the amount of data processing that is intended to be performed by the monitoring device 12.

In the embodiment of the monitoring device 12 illustrated in FIG. 2, four wireless interface units 40, 42, 44, 46 are connected to the data bus 36 so as to be in communication with the central unit 34. Each wireless interface unit 40, 42, 44, 46 is a radio transceiver capable of transmitting at a specific frequency or frequencies and using a specific protocol to interface with a respective one of the wireless networks 16, 18, 22, 24. As shown in FIG. 2, each wireless interface unit 40, 42, 44, 46 has its own antenna 48, 50, 52, 54. In alternate embodiments, antennas may be shared among multiple wireless interface units 40, 42, 44, 46, particularly if two of the wireless networks 16, 18, 22, 24 operate at the same or substantially the same frequency. In that case, a switch may be used to regulate which wireless interface unit 40, 42, 44, 46 is using the antenna or antennas. One advantage of using separate wireless interface units 40, 42, 44, 46 is that standard, off-the-shelf components may be used. As one example, a Texas Instruments Chipcon cc2420 transceiver may be suitable for an IEEE 802.15.4 wireless interface unit.

Also connected to the data bus 36 are a number of sensors and elements of various types. The precise number and type of sensors and elements in the monitoring device 12 may vary from embodiment to embodiment, and some embodiments may be adapted for particular monitoring tasks for which only certain sensors are required. Several general types of sensors and elements are found in the medical monitor: position and orientation sensors 60, input/output control elements 70, ambient condition sensors 80, and physiological sensors 90; however, some sensors and elements may serve more than one purpose. Generally speaking, data gathered by the various sensors and elements within the monitoring device may be used for treatment purposes, monitoring purposes, research purposes, or for any other purpose, although the description that follows may focus on certain specific examples.

Position and orientation sensors 60 establish the position of the monitoring device 12 and its orientation. In the embodiment of FIG. 2, the position and orientation sensors include a global positioning system (GPS) receiver 62, a gyroscope 64, and an accelerometer 66. In addition to locating the monitoring device 12, these may serve a diagnostic purpose as well. As an example, GPS data establishes the device's location and altitude, which can be used diagnostically and to determine monitoring needs; in one embodiment, if the GPS data indicates that the patient has suddenly increased 4,000 feet in altitude, the monitoring device 12 may activate an ECG to determine whether the patient's heart has been affected by the change in altitude. As another example, the accelerometer 66 and gyroscope 64 indicate the device's orientation. A sudden change in orientation, as detected by the accelerometer 66 and gyroscope 64 may indicate that the patient has passed out or fallen down suddenly.

Input/output control elements 70 allow the monitoring device 12 to be configured, maintained, programmed, and connected directly to other devices or peripherals. Included in the exemplary group of input/output control elements 70 of FIG. 2 are the device's display and controls 72, an audio controller 74, and an I/O port 76 or group of I/O ports.

The display and controls 72 may be any conventional display and controls known in the art. For example, in a simple embodiment, the display could be a simple LCD display adapted to display the device status and, optionally, some or all of the data being gathered by the monitoring device 12. In other embodiments, the display may be a color LCD screen adapted to display most or all of the data being gathered. Additionally, touch-screen technology could be provided so as to allow the user to input commands.

The audio controller 74 is adapted to output auditory alerts, announcements, and notifications. Depending on the embodiment, the audio controller 74 may also be adapted to digitize and process speech so as to accept voice commands. Additional uses for and functions of the audio controller will be described below in greater detail. If a monitoring device 12 is equipped with an audio controller 74, then the monitoring device 12 would generally also be equipped with internal speakers and an internal microphone in order to support the functions of the audio controller 74.

The I/O port or group of I/O ports 76 allow the monitoring device 12 to communicate via a wired connection with other devices. This may be useful, for example, when configuring the monitoring device 12, when downloading data from the monitoring device 12, and in situations where no wireless networks are available. Depending on the embodiment, any type and number of I/O ports 76 may be included in the monitoring device 12, including universal serial bus (USB) ports, mini-USB ports, FireWire ports, RS-232 serial ports, and Ethernet ports. Additionally, in some embodiments, the monitoring device 12 may be equipped with a wireless I/O port, such as an infrared communication port.

The ambient condition sensors 80 allow the monitoring device 12 to sense the ambient conditions around the monitoring device 12 and the patient and, in particular, to sense ambient conditions that might be dangerous for the patient. Shown in FIG. 2 are an ambient temperature sensor 82, a vibration sensor 84, and an ambient light sensor 86. The vibration sensor 84 may be an accelerometer, and, in some embodiments, the accelerometer 66 may be used as the vibration sensor 84; however, the vibration sensor 84 is shown as a separate component in FIG. 2 in order to convey the full scope of its functions.

Accelerometers, in particular, may have many different functions in the monitoring device 12, and if multiple accelerometers are provided, each one may be adapted for a particular function. For example, if a monitoring device 12 is worn consistently during daily activity and the patient or user is injured during wear, accelerometer data can be used to gauge the severity of the impact or injury. Accelerometers can also be used for body position detection, as was noted briefly above, and for body position monitoring. Additionally, in some embodiments, accelerometer data may be used to “learn” a patient or user's usual daily movements, so as to determine if the user is making abnormal or labored movements and to identify movements or movement habits that may cause injury or exacerbate a pre-existing condition.

In addition to the components delineated above as ambient condition sensors 80, certain other sensors may be used as ambient condition sensors 80 if desirable or necessary, and the data developed may be used for treatment purposes as well as for research purposes. For example, in some cases, the audio controller 74, equipped with an internal microphone, could be an ambient noise sensor to detect noises that are extraordinarily loud or otherwise out of the ordinary. As an example of that, if the monitoring device 12 is worn by a soldier, it might record gunshots secondary to that soldier being injured. The nature and volume of the gunshot sound, coupled with other data, such as information on the wireless network topology and monitoring device 12 location, may allow interested parties to reconstruct the location of the shooter.

There are a plethora of physiological sensors 90 that may be included in the monitoring device 12, only a few of which are shown in FIG. 2. As was described briefly above, the number and type of physiological sensors 90 in the monitoring device 12 will depend on the particular application for which the monitoring device 12 is designed and other considerations, such as the total desired size and weight, the total desired power consumption, and the total desired complexity of the device 12. Among the physiological sensors 90 that are illustrated are a blood pressure monitor 92, an ECG 94, an EEG 96, a pulse oximeter 98, a body temperature sensor 100, and a skin galvanic sensor 102. (The skin galvanic sensor 102 is a component capable of sensing the electrical potential of the skin. That would allow the monitoring device 12 to detect, for example, whether a patient is sweating.)

Other exemplary physiological sensors 90 that may be included in the monitoring device are an ultrasound device, such as a MEMS-based ultrasound transducer to detect chest wall motion, an end-tidal carbon dioxide detector, a non-invasive glucose detector, and an anemia detector.

In some embodiments, the monitoring device 12 may also include actuators or actuator controllers 104 in order to interact with or drive other medical devices. For example, in some embodiments, the monitoring devices 12 may include an actuator to drive an automatic infusion pump. As was noted briefly above, the actuators or actuator controllers 104 would allow the monitoring device 12 to take an active role in the delivery of medical interventions and care.

There are also certain technologies that may be incorporated into the monitoring devices 12 in order to facilitate locating and tracking them, either for patient monitoring purposes or for asset tracking purposes. The GPS receiver 62 may be used for that task in some or most locations. However, where GPS reception is not available, other technologies may be used. For example, the monitoring device 12 may be equipped with an active or passive RFID tag 106 (an active RFID tag 106 is shown in FIG. 2). Additionally or alternatively, the monitoring devices 12 may include ultrawideband (UWB) locating devices. In general, GPS receivers, RFID tags, and UWB locating devices are all types of spatial location sensors, any sort of which may be included in embodiments of the invention.

In order to power its components and allow portability, the monitoring device 12 also includes a power system 150. The power system 150 would typically comprise a battery of sufficient capacity to power the monitoring device 12 for a clinically useful period of time, along with means for allowing the monitoring system 12 to draw standard household and commercial power. The battery may be any type of battery, including disposable batteries and rechargeable batteries. If the battery is rechargeable, then the power system 150 would generally allow the battery to be recharged while installed in the device.

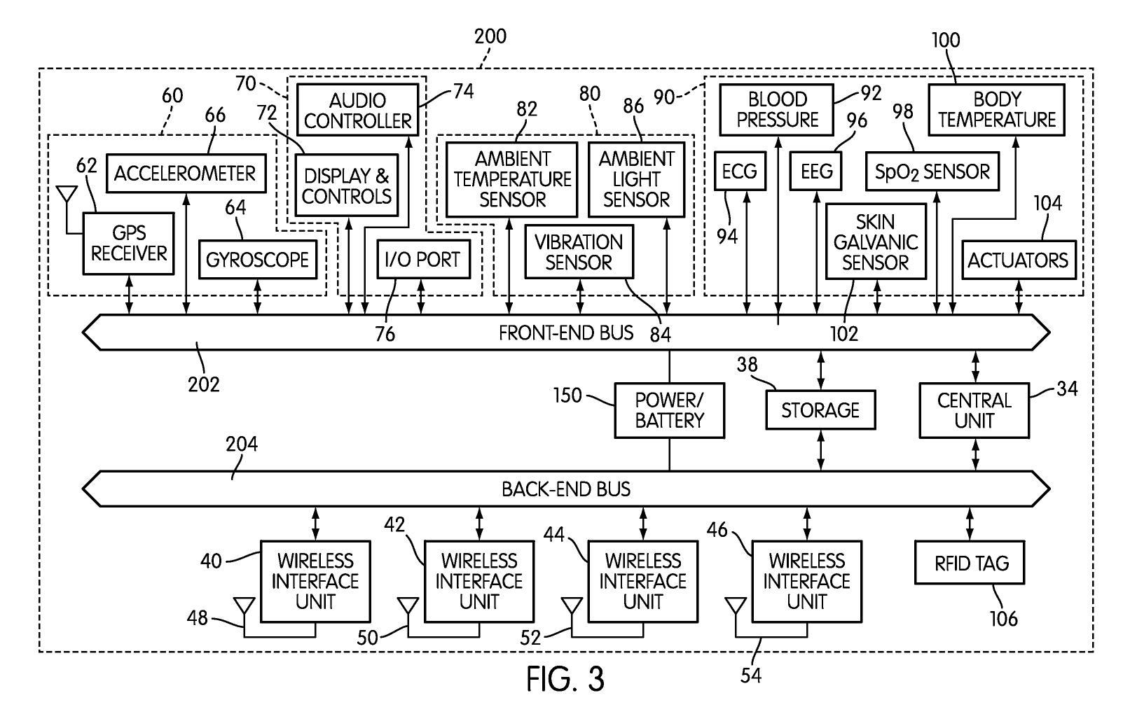

The internal architecture of the monitoring device 12 may vary from embodiment to embodiment. As one example, the monitoring device 12 includes a single data bus 36. However, in some embodiments, it may be advantageous to provide separate data buses for the sensors and the wireless interface units. FIG. 3 is a schematic illustration of another embodiment of a monitoring device 200. The monitoring device 200 is similar in many respects to the monitoring device 12; therefore, components not described here may be assumed to be the same as or substantially similar to those of the monitoring device 12.

In monitoring device 200, there are two data buses, a front-end bus 202, to which most of the sensors and elements 60, 70, 80, 90 are connected, and a back-end bus 204, to which the wireless interface units 40, 42, 44, 46 are connected. The central unit 34, storage 38, and power system 150 are connected to both data buses 202, 204 so as to supply power to and be in communication with all of the components. Thus, each bus 202, 204 can be configured for the type and bandwidth of data that it handles.

Altogether, the architecture of the monitoring devices 12, 200 is similar in many respects to that of a general-purpose computer. Therefore, in some embodiments, the monitoring devices may be simplified, such that they comprise only the sensors and one or more of the wireless interface units used for communication. In those embodiments, substantially all processing would be done by the remote monitoring and control station 14 or by another remote general-purpose computer. In the simplest embodiment, the medical monitoring devices may comprise little more than one or more independent sensors that transmit wirelessly. An advantage of this sort of embodiment is that the individual monitoring devices are inexpensive and easily maintained.

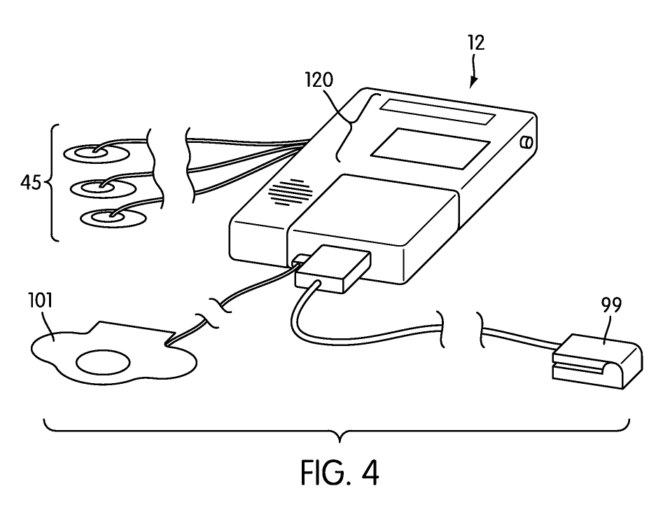

Externally, the monitoring devices 12, 200 may have any size, configuration, or features that are conducive to portability. FIG. 4 is a perspective view of one embodiment of a monitoring device 12, 200. In the view of FIG. 4, a display and set of controls 120 are visible, as are leads for several sensors. In particular, ECG leads 45, a pulse oximetry clip 99, and body temperature sensor 101 lead are shown in FIG. 4.

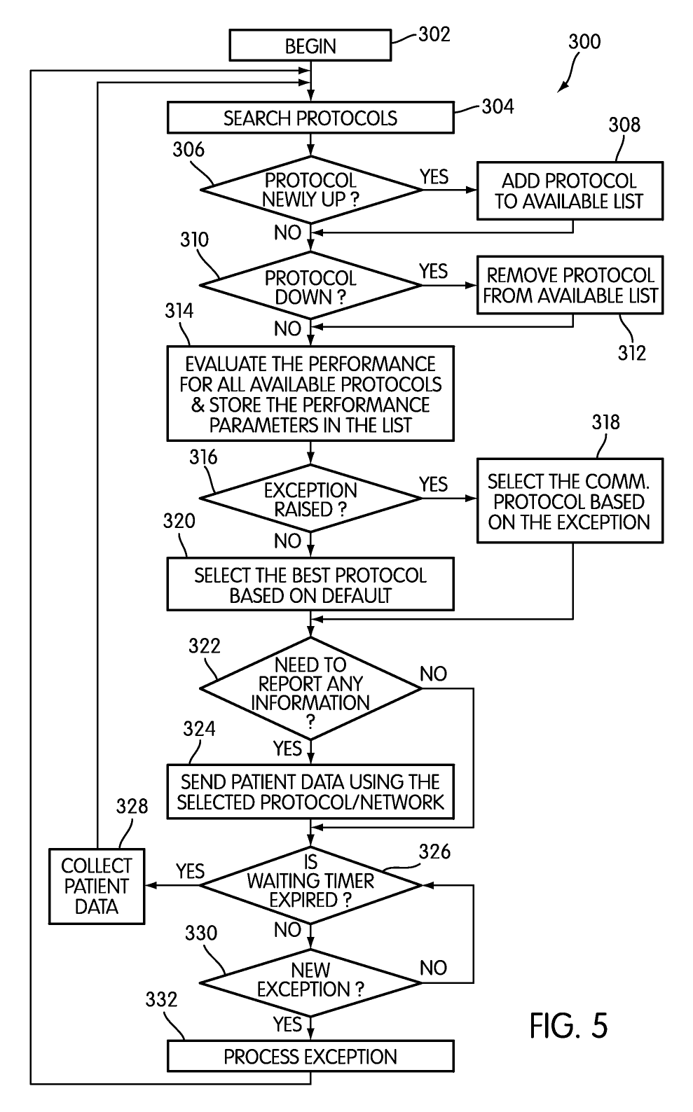

FIG. 5 is a flow diagram illustrating a basic method 300 for collecting data from the various sensors and elements 60, 70, 80, 90 and selecting one or more of the wireless networks 16, 18, 22, 24 through which to transmit the data to the remote monitoring and control station 14. Method 300 begins at 302 when the monitoring device 12, 200 is powered on, and continues with 304. In task 304, once the monitoring device 12, 200 is powered on, it begins a search for available wireless networks. This would generally be done in a conventional and network/protocol specific manner for each network, frequency, or protocol. Method 300 continues with task 306, a decision task.

In task 306, if any active wireless networks or protocols are found (task 306: YES), then method 300 continues with task 308 those networks or protocols are added to an available network/protocol list kept by the monitoring device 12. If no new networks or protocols are found (task 306: NO), and once any new networks have been added to the available list, method 300 continues with task 310.

Task 310 is a decision task. If a particular network or protocol that was previously on the available network/protocol list was not found in task 304 (task 310: YES), then that network or protocol is removed from the available list in task 312 before method 300 continues with task 314. Otherwise (task 310: NO), method 300 continues directly with task 314.

In task 314, the monitoring device 12, 200 evaluates the performance of each available protocol or network. For example, it may determine the signal quality and bandwidth of each available protocol or network, the degree of traffic congestion for the protocol or network, the data packet loss rate, the power requirements for transmission, and the estimated delivery time for data sent using each protocol or network. Those performance parameters are then stored in the available protocol/network list.

Following those power-on protocol and network detection tasks, method 300 continues with a loop of tasks that continues until the monitoring device 12, 200 is shut down. First in that loop of tasks is task 316, in which the monitoring device 12, 200 determines whether an exception has been raised. The term “exception,” as used here, refers to any event or circumstance requiring the monitoring device 12, 200, or one of its components, to take a specific action. Exceptions, in this context, may refer to either an event internal to the monitoring device 12, 200 (e.g., a low battery, a component failure, or a command given to the monitoring device 12, 200) or a patient event (e.g., a sensor reading grossly outside of normal limits, or a change in sensor readings beyond a predetermined threshold that may indicate a positive or adverse change in a patient's condition).

As a more specific example of a patient exception or event, in the description above, it was noted that after a rapid change in altitude, as recorded by the GPS receiver 62, it might be desirable to activate the ECG 94 to check the patient's heart rhythm and rate. Thus, a rapid change in altitude may raise an exception so that appropriate action can be taken to activate the ECG 94.

It should also be understood that data from one or more of the sensors and elements can be compared and, if that data disagrees by more than a predetermined threshold, then a device exception indicating device failure can be raised. For example, a patient's heart rate can be determined by examining either ECG data or pulse oximetry data. In some embodiments, ECG and pulse oximetry data on the patient's heart rate may be compared. If that data disagrees by more than a predetermined acceptable threshold, then an exception can be raised.

If an exception is raised in task 316 (task 316: YES), then method 300 continues with task 318, in which a protocol or network is selected to convey the exception information to the remote monitoring and control station 14. As was noted briefly above, the protocol or network used to communicate particular information may be selected based on the type of data and other factors.

Generally, the performance parameters that are determined and stored in task 314 are used to select a protocol or network for communicating a particular exception. For example, if the exception is one that does not require significant bandwidth to communicate (e.g., device failure), then a protocol or network that has a smaller bandwidth could be selected. If the exception requires higher bandwidth to communicate, then a higher bandwidth protocol or network may be selected. Additionally, if there are several available networks, then the selection may be based on the signal strength for each protocol or network. For example, if two high-bandwidth protocols or networks are available, the monitoring device 12, 200 may choose the protocol or network with the stronger or better quality signal. However, the monitoring device 12, 200 may also be configured such that if the data to be transmitted would normally require high bandwidth, but only a low-bandwidth network or protocol is available, the data that is transmitted is selected, compressed, or pared down to transmit what can be transmitted over that low-bandwidth network. For example, transmitting a full ECG generally requires a reasonably high bandwidth. However, if no high-bandwidth protocol or network is available, the monitoring device 12, 200 may be programmed to send a simpler, shorter message (e.g., “arrhythmia warning” or its equivalent in a numeric or other code) over a low-bandwidth network or protocol, instead of transmitting the patient's full ECG.

If no exception is raised in task 316 (task 316: NO), method 300 continues with task 320, and a default protocol or network is selected based on the available list. Method 300 then continues with task 322, a decision task.

In task 322, if any patient data has been gathered but has not yet been transmitted (task 322: YES), that patient data is sent to the remote monitoring and control station 14 using the protocol or network chosen in tasks 318 and 320 in task 324. If no data exists to be transmitted (task 322: NO), then method 300 continues with task 326, another decision task.

Typically, while in operation, the monitoring devices 12, 200 will collect patient data continuously at predetermined intervals. Those predetermined intervals may be short (a few milliseconds or shorter between readings) or they may be long (minutes, seconds, or hours between readings). As one example, the monitoring devices 12, 200 may transmit data every 0.5 seconds. Generally, a timer would be set after data is read or an exception occurs, and after that predetermined interval expires, the monitoring device 12, 200 would check for another exception and/or more data to send. In task 326, if the interval timer has expired (task 326: YES), method 300 continues with task 328, in which patient data is collected. Following task 328, control of method 300 returns to task 304.

If the timer has not expired (task 326: NO), control of method 300 passes to task 330, another decision task in which it is determined whether there has been any new exception. If there has been an exception (task 330: YES), method 300 continues with task 332 and the exception is processed. If there has been no new exception (task 330: NO), control of method 300 returns to task 326. The overall effect of tasks 326-332 is to create the predetermined interval or pooling period between data transmissions, and to keep the monitoring device 12 in a “sleep” or low-power state for the majority of that predetermined interval unless an exception occurs.

As shown in FIG. 5, the monitoring device 12, 200 continues with method 300 returning to task 304 unless it is powered down or instructed to terminate method 300 (e.g., by an exception generated because the user issues a command or by a device exception that requires shutdown). The monitoring device 12, 200 searches, evaluates, and selects a transmission protocol or network essentially each time data exists to be transmitted, which, at least in part, provides for the seamless communication described above. Should the monitoring device 12, 200 be unable to transmit a particular packet of data, or should a packet of data be lost, an exception would be raised, and the response to that exception could be any one of a number of actions. For example, the monitoring device 12, 200 could be programmed to retransmit the data in question. Ultimately, if several transmission attempts fail and no reliable wireless network or protocol can be found, the monitoring device 12, 200 may store the data for transmission when a wireless network or protocol does become available.

As described above, the particular response to any exception may depend on the nature of the exception and on other factors. The response to an exception may involve any number of tasks. Depending on the embodiment, for some exceptions, the monitoring device 12, 200 may prompt the user or medical professional to correct the condition.

One of the difficulties with monitoring devices in general is that it can become difficult to determine which exceptions or device alarms require immediate attention from medical personnel and which can wait. Particularly if a plurality of devices is in use monitoring different patients simultaneously, alarms may be sounded often, sometimes so often that medical personnel become inured to them and lose a sense of urgency.

Generally speaking, when a sensor is incorrectly positioned, moves out of its correct position, or loses contact with the patient in some other way, the data collected by that sensor will become erratic and an exception will be raised. In some cases, that erratic data could be read (falsely, in most cases (to indicate that the patient's condition has worsened.

However, in some embodiments, the monitoring devices 12, 200 may be programmed to handle a sudden change in a sensor's data that brings the data far outside the expected ranges by assuming that the sensor has been disconnected or has lost contact with the patient. In fact, this feature may be implemented in monitoring devices other than the monitoring devices 12, 200, and monitoring devices that include this feature may or may not include all of the features described above with respect to the monitoring devices 12, 200.

Thus, when the central unit 34 perceives that a sensor or element's data has suddenly moved outside of a predetermined range, instead of raising an exception indicating that the patient's condition has worsened, the central unit 34 could prompt the patient or medical personnel to reposition or reattach the sensor in question. For example, if a pulse oximeter falls off of a patient's finger, the patient might be given an auditory prompt to “please reattach the pulse oximeter to your finger.” That prompt may be followed by additional auditory instructions on how to reattach the pulse oximeter, and, if the device in question includes a visual display, the display may present the user with a graphic, animated graphic, video clip, or another form of tutorial illustrating how to reattach the pulse oximeter. When a new prompt begins, all other audio and visual output on the monitoring device may be halted temporarily. Prompts can be provided in different or multiple languages, depending on the embodiment and the needs of the patient and medical professionals.

Auditory and/or visual prompts may continue until data within the expected ranges is received from the sensor. Prompts may be given more frequently at first and then at increasingly longer time intervals. However, if the sensor in question is important to the operation of the monitoring device, then prompts may be given more frequently than for a sensor of somewhat less importance. Once the patient or professional reattaches the sensor in question, the prompts would typically be terminated. Moreover, in order to save power, if the patient or professional fails to respond to repeated prompts, then the prompts may cease and the display and audio system may be powered down. Ultimately, if a patient does fail to respond, then an alarm may be sounded at a remote central monitoring station (e.g., by the remote monitoring and control station 14) so that medical personnel can attend to the condition. By giving the patient an opportunity to correct the problem first, this method would likely reduce the number of alarms to which medical personnel are forced to respond.

In order to avoid generating false alarms when a sensor is deliberately disconnected for repositioning or routine maintenance, a prompt to reattach the sensor may not be issued for some predetermined time after the sensor data falls outside of the expected limits. The ability to temporarily or permanently disable the prompts may also be provided.

The tasks involved in offering prompts may be performed by the medical device itself, by the remote station, or by a combination of the two. For example, if a more sophisticated algorithm is necessary to determine whether a sensor has become disconnected, that algorithm could be performed on the remote station, rather than on the monitoring device. The actual prompts could be stored within the monitoring device or within the remote station, depending on the sophistication and storage space available in the monitoring device. If the prompts are stored on a remote monitoring device, then they may be transmitted to the monitoring device in digital or analog form. (For example, audio prompts could be transmitted in analog form using a conventional AM or FM transmitter.)

The mechanism for handling prompts may be different or distinct from the remote station that otherwise processes data from the medical monitoring devices, and other devices may be involved in the prompt delivery. For example, the prompt delivery functions could be invested in a centralized prompt delivery unit. When a prompt is to be sent, the user could be instructed to turn their room television to a particular channel or tune their room radio to a particular frequency to receive the prompt. Other devices may also be provided for prompt delivery. Thus, the actual monitoring device may be largely removed from the process of actually delivering the prompt, which may be advantageous in some embodiments, particularly with monitoring devices of limited capabilities.

Some prompts may have nothing to do with individual sensors. For example, a monitoring device may monitor a patient's location and inform that patient to “return to the emergency department,” or to another specified location, when medical professionals are ready to treat them. In that way, the monitoring devices may act as a specialized paging system.

In some embodiments, monitoring devices may be equipped to display other types of non-urgent or non-medical audio and video in order to occupy a patient. Generally speaking, the same hardware and components that are used for medical monitoring could be used for non-medical purposes as well. For example, a monitoring device may be equipped to play music or to allow the patient to play video games. If such non-urgent audio and video is being played when a prompt is to be issued, the prompt would typically pre-empt the non-urgent audio and video.

Ultimately, using system 10 and method 300, patients can move seamlessly from the ambulance to the hospital, from the hospital to the rehabilitation center, and from the rehabilitation center back to home, car, and work using the same medical and monitoring device 12, 200, because that device automatically takes advantage of whatever wireless networks or protocols are available and selects the most suitable wireless network or protocol for each transmission. System 10 and method 300 thus provide substantially uninterrupted communication between the remote monitoring and control station 14 and the monitoring devices 12, 200 even in heterogeneous network environments in which the user is moving between locations covered by different types of wireless protocols and networks. Moreover, the monitoring device 12, 200 may be selective in its use of its various sensors and elements, activating some of them only when necessary. Furthermore, it may actively recognize improperly positioned sensors and other conditions and prompt the user to correct those conditions.

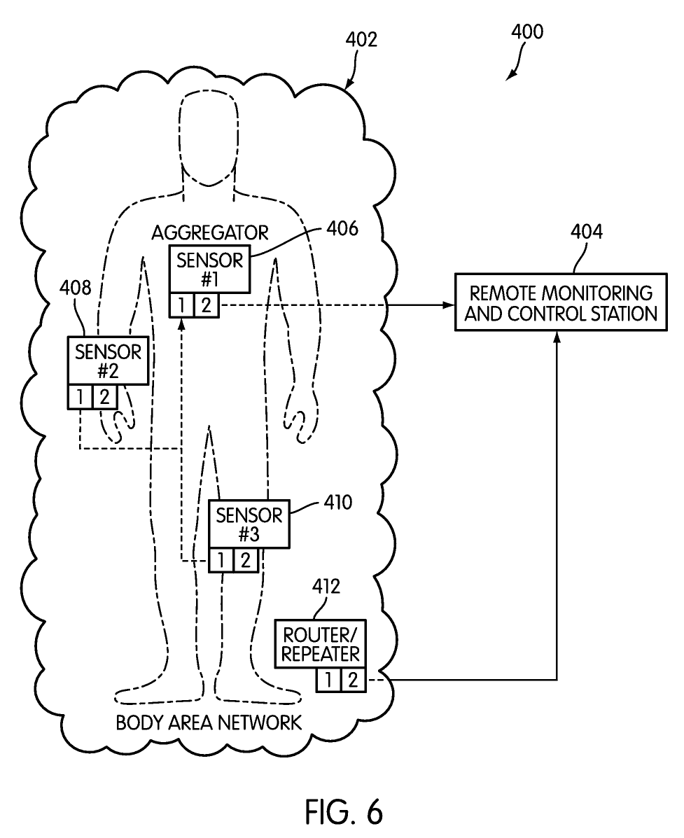

The description above assumes that the sensors and elements that comprise the monitoring device 12, 200 are essentially located in one unitary “box.” With the flexibility afforded by wireless communication, that need not be the case. FIG. 6 is an illustration of a system, generally indicated at 400, according to yet another embodiment of the invention.

In system 400, one or more sensors, usually consuming lower power and less processing power than the multifunction monitoring devices 12, 200 described above, and typically not physically connected with one another, communicate with each other wirelessly to form a body area network 402. In the body area network 402, at least one of the sensors is chosen as an aggregator, and is responsible for communicating aggregated data from all of the sensors to a remote monitoring and control station 404, and for communicating any commands or instructions from the remote station 404 to the other sensors.

Specifically, in system 400 of FIG. 6, there are three individual sensors 406, 408, 410. Each of those sensors would generally be a scaled down, single-purpose version of the monitoring devices 12, 200 described above. The sensors 406, 408, 410 may be any of the types of sensors described above with respect to the monitoring devices 12, 200. Typically, each sensor 406, 408, 410 would include the actual sensing hardware, a processor or other form of central unit to collect and process the data, and one or more wireless interface units. (In the sensors 406, 408, 410 of FIG. 6, the wireless interface units are schematically labeled “1” and “2.”) In the broadest terms, the wireless interface units are radios adapted to transmit and receive data. If a sensor 406, 408, 410 includes more than one wireless interface unit, one is typically dedicated to local or short-range communication with the other sensors, and another may be dedicated to long-range communication with a remote monitoring and control station 404 or to other types of communication outside of the body area network 402. In order to participate in the body area network 402, each sensor would have at least a short-range wireless interface unit; long-range units are optional except in sensors 406, 408, 410 that are to serve as aggregators. In general, as used herein, the terms “short range” and “local” refer to an ability to transmit and receive at least within a volume of space typical of the volume of space occupied by a human body, whereas the term “long range” refers to an ability to communicate outside of that volume of space.

In one embodiment, the wireless interface units may be physically different, adapted to transmit using different protocols, and/or at different power levels. For example, one wireless interface unit could be adapted to communicate via the ZigBee or Bluetooth protocols, while another may be adapted to communicate using WiFi or WMTS.

In other embodiments, the wireless interface units may be physically identical but configured differently. For example, they could be two WiFi interface units adapted to communicate on different data channels, and with different power levels. Alternatively, two WiFi interface units could communicate using different encryption schemes, different compression schemes (e.g. lossy vs lossless), different reliability schemes (e.g., different retransmission rates), or a combination of any of the above. It should be understood that “short range” and “long range” wireless interface units may comprise physically identical units that are merely configured differently.

In system 400 of FIG. 6, sensors 406, 408, and 410 have two wireless interface units. Furthermore, sensor 406 is the designated aggregator, and has the responsibility of communicating with the remote monitoring and control station 404. Thus, sensors 408 and 410 transmit their data to sensor 406, which bundles it and transmits it to the remote monitoring and control station 404. The remote monitoring and control station 404 may have essentially the same features as the remote monitoring and control station 14.

When the sensors 406, 408, 410 of system 410 are turned on, they synchronize with one another and select an aggregator. One consideration in system 400 is which sensor 406, 408, 410 becomes the aggregator. The aggregator would be a sensor with a wireless interface unit capable of communicating with the remote monitoring and control station 404. Beyond that basic communication capability, the aggregator may be the sensor with the longest battery life, it may be the sensor with the most processing power, or it may be the sensor most capable of communicating with the remote monitoring and control station 404. Furthermore, as will be described below in more detail, the identity of the aggregator may change over time as conditions change.

In system 400, as was described above, any number of sensors 406, 408, 410 may be equipped to communicate with the remote monitoring and control station 404, and, in fact, as few as one sensor may have that capability. However, if multiple sensors 406, 408, 410 have the capabilities to communicate with the remote monitoring and control station 404, certain advantages may be realized.

Therefore, in embodiments of the present invention, and in cases in which more than one sensor 406, 408, 410 has the capabilities to communicate with the remote monitoring and control station 404, the identity of the aggregator may change from time to time. Sensor conditions, environmental conditions, the nature of the data to be transmitted, and control signals or requests from the remote monitoring and control station 404 may be taken into account in determining which sensor 406, 408, 410 acts as the aggregator and which specific wireless protocols are used to communicate with the remote monitoring and control station 404. When more than one aggregator is selected among the group of sensors, data can be transmitted via multiple aggregators simultaneously. This redundancy provides the benefit of added reliability and may also aid in seamless communication between the body area network 402 and the remote monitoring and control station.

Many of the methods described above with respect to selecting particular wireless networks and protocols for the monitoring devices 12, 200 based on the available wireless networks and their properties apply equally to system 400. Additionally, other factors that may specifically be used to select an aggregator include sensor processing power, bandwidth, power consumption, battery power level, transmission power, and sensor precision or reliability, among others. Generally speaking, the aggregator may be chosen based on the best available protocol with which to communicate a particular piece of data to the remote monitoring and control station 404 at any particular time. As with the embodiments described above, to the extent that the aggregator and the communication protocols change, the changes are most advantageously seamless.

One particular challenge in managing a system such as system 400 lies in managing the available battery power in the sensors 406, 408, 410. As those of skill in the art will realize, the sensor 406, 408, 410 chosen as the aggregator may use more power than the other sensors, and thus, may drain its battery faster. Thus, one particular sensor condition that may be used to select an aggregator is power condition in the various sensors 406, 408, 410, and particularly battery level. For example, the aggregator could initially be chosen as the sensor 406, 408, 410 with the most battery power remaining. When that sensor, sensor 406 in FIG. 6, reaches a certain threshold of battery power remaining, for example, 50% or 25% power remaining, another aggregator (e.g., sensor 410) may be chosen, and the previous aggregator may return to a sensor-only role.

However, power considerations are not always so straightforward. For example, a situation could arise in which one sensor reaches a low battery threshold and hands off aggregator functions to another sensor that actually has a lower battery level, but also has components that draw less power, such that the sensor with the lower battery level will actually last longer than the sensor that originally acted as aggregator. Ultimately, it is advantageous in system 400 if power usage and other functions are distributed across the system as evenly as possible.

In other situations, the nature of the medical data to be transmitted may be the controlling factor. For example, a sensor of less complexity and with lower power consumption may be in a body area network 402 with a sensor of higher complexity. The less complex sensor may be used as the aggregator unless it is necessary for the higher complexity sensor to transmit data requiring more bandwidth or processing power. For example, an ECG and a pulse oximeter may be in a body area network 402 together, and the pulse oximeter may act as the aggregating sensor in most circumstances, unless the ECG actually needs to transmit a substantial amount of ECG data.

In addition to the considerations explained above with respect to selecting an aggregator, the sensors 406, 408, 410 may also be programmed with a designated order of priority that determines which sensor 406, 408, 410 becomes the aggregator. Such a designated order of priority may be used instead of or in addition to other technical selection criteria. For example, an order of priority could be designated such that if one of the sensors 406, 408, 410 is a cardiac monitor, that sensor becomes the aggregator so long as its other selection criteria are reasonable compared to those of the other sensors 406, 408, 410. That is, if the sensor highest in the order of priority is grossly low on power, if signal quality on the wireless networks it can access is low, if it does not have the bandwidth or processing capability to transmit a certain type of data, or if there is some other reason why the highest-priority sensor cannot perform the task, the designated order of priority may be overridden. In that case, the sensor next in the order of priority may be chosen. If two sensors equal in priority are vying for the functions of aggregator, the selection algorithm may decide between them based on the current status of each.

In the embodiment of FIG. 6, the non-aggregating sensors 408, 410 communicate with the aggregator (sensor 406) only. That need not always be the case. So long as they communicate with the aggregating sensor as necessary, the other sensors 408, 410 in a body area network may communicate with other devices and for other purposes. For example, as was described above with respect to other monitoring devices 12, 200, the sensors 406, 408, 410 may communicate directly with PDAs or other mobile devices.

FIG. 6 illustrates another optional part of a body area network 402. A body area network 402 may include non-sensing elements that communicate with or receive data from the sensors 406, 408, 410. Essentially any element that can communicate with the sensors 406, 408, 410 may be included in the body area network 402. However, two categories of devices that may be particularly useful are routers and repeaters. A router/repeater element 412 is schematically illustrated in FIG. 6.

In general terms, a repeater is an element that receives a signal and re-transmits it, usually at a higher power, allowing the signal to cover longer distances without degradation. A router is an element that handles routing and forwarding tasks in a network; i.e., the process of selecting paths in a network along which to send data, and the passing of data from its source toward its ultimate destination through a number of intermediate nodes. In FIG. 6, the router/repeater 412 is shown as being in communication with the remote monitoring and control station 404, although that need not be the case in all embodiments, as routers and repeaters may also be used to communicate with other types of devices.

If non-sensing network elements such as router/repeater 412 are included in the body area network 402, they may be worn by the patient, in which case, they may be low-power devices essentially similar in transmission capabilities to the sensors 406, 408, 410 themselves. Alternatively, depending on the embodiment and the use to which they are put, the non-sensing elements may have more transmitting power, more processing capabilities, and more wireless interface units than any of the sensors 406, 408, 412.

While the invention has been described with respect to certain exemplary embodiments, those embodiments are intended to be illuminating, rather than limiting. Modifications and changes may be made within the scope of the invention, which is defined by the claims.

What is claimed is:

1. A wireless medical monitor, comprising:
one or more sensors;
one or more wireless interface units coupled to the one or more sensors, each of the two or more wireless interface units being adapted to transmit data from the one or more sensors using a different transmission protocol; and
wherein the medical monitoring device is adapted to select wireless interface units and to switch between their respective different transmission protocols seamlessly.

2. The medical monitor of claim 1, wherein the one or more sensors comprise one or more of physiological, environmental, or activity sensors.

3. The wireless medical monitor of claim 2, wherein the physiological sensors comprise one or more sensors selected from the group consisting of electrocardiogram sensors, electroencephalogram sensors, pulse oximetry sensors, heart rate sensors, respiration sensors, blood pressure sensors, skin galvanic sensors, blood glucose sensors, anemia detectors, and body temperature sensors.

4. The wireless medical monitor of claim 2, wherein the activity sensors comprise one or more spatial location sensors.

5. The wireless medical monitor of claim 2, wherein the environmental sensors are selected from the group consisting of ambient temperature sensors, ambient light sensors, and ambient vibration sensors.

6. The wireless medical monitor of claim 1, wherein the one or more sensors are independent of one another, each having one or more of the wireless interface units coupled thereto, the one or more independent sensors being in wireless communication and cooperation with one another.

7. The wireless medical monitor of claim 6, wherein the one or more sensors are adapted to select an aggregating sensor from amongst themselves, the aggregating sensor being operative to aggregate data from the other sensors and to communicate that data to an external station.

8. The wireless medical monitor of claim 7, wherein the one or more sensors are further adapted to change which one of the one or more sensors acts as the aggregating sensor based on environmental conditions, sensor conditions, or instructions from the external station.

9. The wireless medical monitor of claim 1, further comprising a central unit connected between the one or more sensors and the one or more wireless interface units, the central unit being adapted to process data from the one or more sensors and to select one or more of the wireless interface units to transmit the data from the one or more sensors.

10. The wireless medical monitor of claim 9, further comprising one or more input/output elements.

11. The wireless medical monitor of claim 10, wherein the input/output elements comprise one or more elements selected from the group consisting of input controls, displays, I/O ports, audio controllers, and voice input controls.

12. A medical monitoring system, comprising:
one or more medical monitors, each of the one or more medical monitors comprising one or more sensors and one or more wireless interface units, each of the wireless interface units being configured and adapted to transmit using a different wireless protocol, the medical monitors being adapted to select one of the wireless protocols for transmission based on one or more of environmental conditions, sensor conditions, or the nature of the medical data to be transmitted.
a remote monitoring and control station in communication with the one or more medical monitoring devices through one or more wireless networks, the remote monitoring and control station being adapted to display the data from the one or more sensors.

13. The medical monitor of claim 12, wherein the one or more sensors comprise one or more of physiological, environmental, or activity sensors.

14. The medical monitoring system of claim 12, wherein the one or more sensors are independent of one another, each having one or more of the wireless interface units coupled thereto, the one or more independent sensors being in wireless communication and cooperation with one another.

15. The medical monitoring system of claim 14, wherein one of the one or more sensors acts as an aggregating sensor, aggregating data from the other medical sensors and communicating with the remote monitoring and control station.

16. The medical monitoring system of claim 15, wherein the aggregating sensor is selected and, if necessary, re-selected based on one or more of sensor conditions, environmental conditions, the nature of the data to be transmitted, a predetermined order of priority, and control signals from the remote monitoring and control station.

17. The medical monitoring system of claim 16, wherein the sensor conditions comprise one or more of battery levels in the sensors, expected battery life in the sensors, and the relative capabilities of the sensors.

18. The medical monitoring system of claim 12, further comprising one or more non-sensing networking elements.

19. The medical monitoring system of claim 18, wherein the non-sensing networking elements comprise routers or repeaters.

20. A method of transmitting medical data, comprising:
determining the properties of one or more wireless protocols or networks;
collecting the medical data from one or more sensors or elements;
assessing the properties of the medical data as they relate to the properties of the one or more wireless protocols or networks;
based on the assessing, selecting one or more of the plurality of wireless protocols or networks to transmit the medical data; and
transmitting the medical data using the selected wireless protocols or networks.

21. The method of claim 20, wherein the properties of the one or more wireless protocols or networks comprise one or more properties selected from the group consisting of signal quality, bandwidth, data loss rate, power consumption, traffic congestion, and delivery delay time.

22. The method of claim 20, wherein the properties of the medical data comprise one or more properties selected from the group consisting of type of data, data urgency, and transmission bandwidth requirements.
